(19) United States
(12) Patent Application Publication
(10) Pub. No.: US 2008/0261253 A1
(43) Pub. Date: Oct. 23, 2008

(54) Methods and kits for the diagnosis of galactosemia

(75) Inventors: Michael Spain, Austin, TX (US); James P. Mapes, Austin, TX (US); Ralph McDade, Austin, TX (US); Kenneth Pass, Glenmont, NY (US)

Correspondence Address: FOLEY AND LARDNER LLP;SUITE 500, 3000 K STREET NW, WASHINGTON, DC, 20007, US

(73) Assignees: Rules-Based Medicine, Inc.; Health Research Inc.

(21) Appl. No.: 12/007,500

(22) Filed: Jan. 11, 2008

Related U.S. Application Data
(60) Provisional application No. 60/884,827, filed on Jan. 12, 2007.

(51) Int. Cl. G01N 33/543

(52) U.S. Cl. 435/7.94

(57) ABSTRACT

Provided are methods for the detection and diagnosis of galactosemia. The methods are based on the discovery that abnormal levels of selected analytes in sample fluid, typically blood samples, of patients who are at risk are supportive of a diagnosis of galactosemia. At least two new biomarkers for galactosemia are thus disclosed, Eotaxin and MCP-1. Altogether the concentrations these markers, individually, or in combinations with any of Alpha-2 Macroglobulin, Apolipoprotein H, Cancer Antigen 125, Leptin, TNF RII, Alpha-Fetoprotein, IgM, MIP-1 alpha, Ferritin, and IgE provide a sensitive and selective picture of the patient's condition, namely, whether the patient is suffering from galactosemia. Kits containing reagents to assist in the analysis of fluid samples are also described.

### CROSS REFERENCE TO RELATED APPLICATIONS

This application claims priority to U.S. provisional application No. 60/884,827 filed Jan. 12, 2007, the disclosure of which is incorporated herein by reference in its entirety.

### FIELD OF THE INVENTION

Methods, kits and reagents for detection and/or diagnosis of galactosemia.

### DESCRIPTION OF THE RELATED ART

Galactosemia is an inherited enzyme disorder transmitted as an autosomal recessive trait. The condition occurs in approximately 1 out of every 60,000 births among Caucasians with different rates in other ethnic groups. There are 3 forms of the disease: galactose-1 phosphate uridyl transferase deficiency—“classic” galactosemia, the most common and most severe form, galactose kinase deficiency, and galactose-6-phosphate epimerase deficiency.

Galactosemia is the inability of the body to metabolize the simple sugar galactose, causing the accumulation of galactose 1-phosphate (G1P) in the body, which accumulation can cause damage to the liver, central nervous system (e.g., brain), kidneys, eyes, and other body systems. Individuals with galactosemia cannot tolerate any form of milk (human or animal) and must carefully watch their intake dairy and any other galactose-containing foods. (Lactose, the predominant sugar in milk, is a disaccharide comprising two sugars, galactose and glucose, bound together).

If an infant, for example, with galactosemia is given milk, derivatives of galactose build up in the infant's system. After drinking milk for a few days, a newborn with galactosemia will refuse to eat and develop jaundice, vomit, become lethargic and irritable, and convulse. The liver typically enlarges and the blood sugar may become low. If the infant continues to feed on milk or milk-products, she will get cirrhosis of the liver, cataracts in the eye (which may result in partial blindness), and mental retardation.

Hence, there is a need for improved screening and diagnosis of patients at risk for galactosemia. The selectivity and sensitivity of current assays for galactosemia, however, are lacking, with the frequency of false positives and false negatives at an undesirable level. Thus, there is a critical need to develop additional biomarkers for early detection of galactosemia.

### SUMMARY OF THE INVENTION

A method for rapid detection and/or accurate diagnosis of galactosemia is provided. The method can be practiced with a determination of the concentrations of one, two or more biomarkers in a patient fluid sample. Elevated (or depressed, as the case might be) levels of the biomarkers, which are statistically different from levels found in “normals” (that is, control subjects not suffering from galactosemia), support a positive diagnosis of galactosemia. Preferably, the method utilizes a panel of analytes or “biomarkers,” up to twelve or more substances found in a sample fluid (e.g., blood spots, whole blood, serum, plasma, or urine), to help support a positive or negative diagnosis of galactosemia. Better than 95% accuracy and preferably up to 99% accuracy in diagnosis can be obtained by the method.

According to the invention a method of diagnosing galactosemia in a human subject, preferably a newborn, suspected of suffering from galactosemia is provided, which method comprises: (a) obtaining a fluid sample from a human subject; (b) determining the concentration of Eotaxin in said fluid sample; (c) deciding if the determined concentration of Eotaxin in said fluid sample is statistically different from that found in a control group of human subjects, whereby a statistically different depressed concentration of Eotaxin supports a positive diagnosis of galactosemia. Typically, the human subject is a newborn. Any one of a number of fluid samples can be tested. Preferably, the fluid sample is selected from blood spots, whole blood, plasma, serum, or urine. It has been discovered that a measured concentration of about 350 pg/mL or below of Eotaxin in the fluid sample supports a positive diagnosis of galactosemia.

In another aspect of the invention a method is provided for diagnosing galactosemia in a human subject, preferably a newborn, suspected of suffering from galactosemia, which method comprises: (a) obtaining a fluid sample from a human subject suspected of suffering from galactosemia; (b) determining the concentration of MCP-1 in said fluid sample; (c) deciding if the determined concentration of MCP-1 in said fluid sample is statistically different from that found in a control group of human subjects, whereby a statistically different depressed concentration of MCP-1 supports a positive diagnosis of galactosemia. It has been found that a measured concentration of about 2.5 ng/mL or below of MCP-1 in said fluid sample supports a positive diagnosis of galactosemia.

Still another aspect of the invention relates to a method of diagnosing galactosemia in a human subject suspected of suffering from galactosemia, comprising: (a) obtaining a fluid sample from a human subject suspected of suffering from galactosemia; (b) determining the concentrations of Eotaxin and MCP-1 in said fluid sample; (c) deciding if the determined concentrations of Eotaxin and MCP-1 in said fluid sample are statistically different from that found in a control group of human subjects, whereby a statistically different depressed concentration of Eotaxin and a statistically different depressed concentration of MCP-1 together support a positive diagnosis of galactosemia.

In a preferred embodiment, the method of the invention further comprises determining the concentration in said fluid sample of at least one of Alpha-2 Macroglobulin, Apolipoprotein H, Cancer Antigen 125, Leptin, TNF RII, Alpha-Fetoprotein, IgM, MIP-1-alpha, Ferritin, IgE, or any combination thereof. It has been found that statistically different concentrations, compared to control levels, of all analytes mentioned above support a positive diagnosis of galactosemia.

Various techniques for assessing the importance of certain biomarkers in arriving at a diagnosis is also described herein. One such technique is a projection of compiled results on a proximity map, whereby the proximity of a subject's determined concentrations to a cluster of other subjects' determined concentrations, who were previously diagnosed as having suffered from galactosemia, contributes to a positive diagnosis of galactosemia. Other techniques include the application of one or more statistical methods (e.g., linear regression analysis, classification tree analysis, heuristic nave Bayes analysis and the like).

Also provided is a kit comprising reagents for determining the concentration in a fluid sample of a panel of analytes including Eotaxin, and MCP-1 and one or more of Alpha-2 Macroglobulin, Apolipoprotein H, Cancer Antigen 125, Leptin, TNF RII, Alpha-Fetoprotein, IgM, MIP-1-alpha, Ferritin, or IgE. The reagents may include antibodies against the members of a given panel of analytes. Furthermore, the reagent may be immobilized on a substrate, which substrate may comprise a two-dimensional array, a microtiter plate, or multiple bead sets.

The methods may further comprise comparing the levels of the one, two, or more biomarkers in a patient's blood with levels of the same biomarkers in one or more control samples by applying a statistical method such as: linear regression analysis, classification tree analysis and heuristic nave Bayes analysis. The statistical method may be, and typically is performed by a computer process, such as by commercially available statistical analysis software. In one embodiment, the statistical method is a classification tree analysis, for example CART (Classification and Regression Tree). Results for a particular patient or subject, whose sample fluid is tested against a panel of biomarkers according to the method, can be projected onto a proximity map.

An article of manufacture is provided which comprises binding reagents specific for at least one of Eotaxin and MCP-1, preferably both biomarkers. More preferably, a kit is provided which comprises binding reagents specific for Eotaxin, MCP-1, Alpha-2 Macroglobulin, Apolipoprotein H, Cancer Antigen 125, Leptin, TNF RII, Alpha-Fetoprotein, IgM, MIP-1-alpha, Ferritin, or IgE. In a preferred embodiment, each binding reagent is immobilized on a substrate. For example, monoclonal antibodies against Eotaxin, and MCP-1 and the other biomarkers described herein are immobilized independently to one or more discrete locations on one or more surfaces of one or more substrates. The substrates may be beads comprising an identifiable biomarker, wherein each binding reagent is attached to a bead comprising a different identifiable biomarker than beads to which a different binding reagent is attached. The identifiable biomarker may comprise a fluorescent compound, a quantum dot, or the like.

Other aspects of the invention will become apparent to those of ordinary skill after considering the detailed descriptions provided herewith.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

FIG. 1 is a projection of a proximity map using principal component analysis (PCA), a technique for simplifying a dataset, by reducing multidimensional datasets to lower dimensions for analysis. PCA linearly transforms data to a new coordinate system such that the greatest variance by any projection of the data comes to lie on the first coordinate (called the first principal component), the second greatest variance on the second coordinate, and so on. PCA can be used for dimensionality reduction in a dataset while retaining those characteristics of the dataset that contribute most to its variance, by keeping lower-order principal components and ignoring higher-order ones. Such low-order components often contain the “most important” aspects of the data. But this is not necessarily the case and depends on the application.

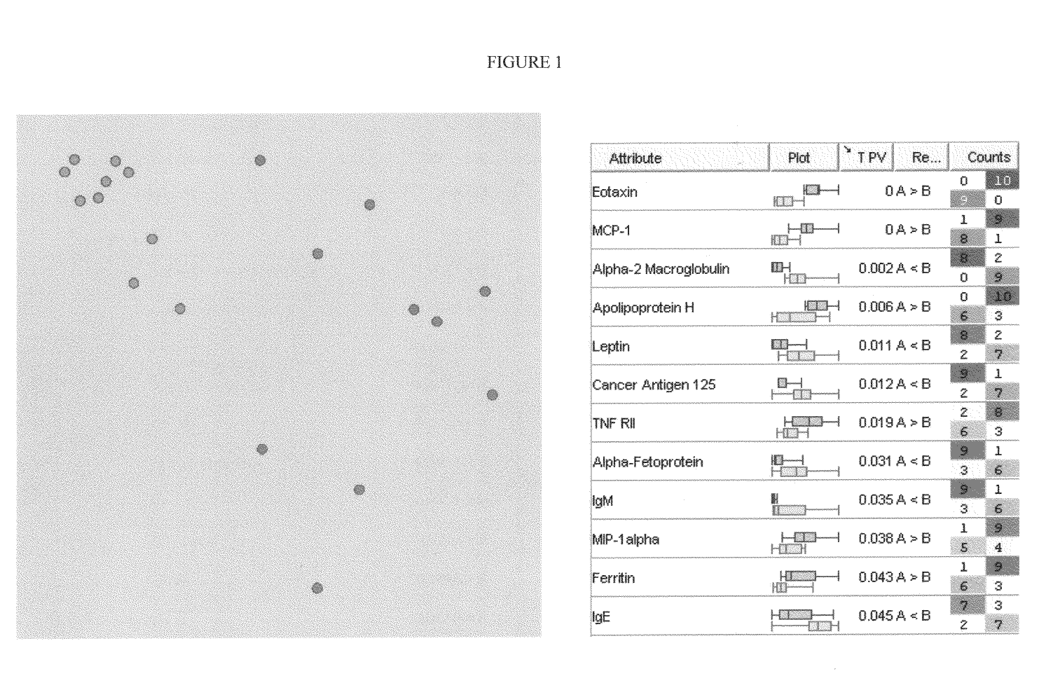

### DETAILED DESCRIPTION OF THE PREFERRED EMBODIMENTS

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. Also, the disclosure of these ranges is intended as a continuous range including every value between the minimum and maximum values.

Provided herein is a multifactorial assay for rapid identification of a galactosemic patient. Identified below are certain sample fluid (e.g., blood) analytes or biomarkers useful in the detection and/or diagnosis of galactosemia. It has been found that Eotaxin is under-expressed in the blood of patients suffering from galactosemia.

Also identified as being useful in the detection or proper diagnosis of subjects suffering from galactosemia are the biomarkers Eotaxin, Monocyte chemotactic protein-1 (MCP-1), and Alpha-2 Macroglobulin, Apolipoprotein H, Cancer Antigen 125, Leptin, Tumor Necrosis Factor Receptor II (TNF RII), Alpha-Fetoprotein, Immunoglobulin M (IgM), Macrophage inflammatory protein 1 alpha (MIP-1-alpha), Ferritin, or Immunoglobulin E (IgE).

The parameters for establishing the significance of one or more biomarkers for the diagnosis of galactosemia are determined statistically by comparing normal or control blood (preferably, e.g., serum or plasma) levels of these biomarkers with blood levels in patients clinically and properly diagnosed as having galactosemia. The statistical data presented below in Table 1 identify certain mean values and accompanying standard deviations for the blood levels of the above-described biomarkers in galactosemic patients and in normals. As a non-limiting example of estimates of significant threshold values in support of a positive diagnosis of galactosemia, the following concentrations are provided: Eotaxin (less than about 365 pg/mL), MCP-1 (less than about 1 ng/mL), Alpha-2 Macroglobulin (greater than about 2.0 mg/mL), Apolipoprotein H (less than about 55 ug/mL), Cancer Antigen 125 (greater than about 5.0 U/mL), Leptin (greater than about 0.57 ng/mL), TNF RII (less than about 5.95 ng/mL), Alpha-Fetoprotein (less than about 330 ng/mL), IgM (greater than about 0.051 mg/mL), MIP-1-alpha (less than about 38 pg/mL), Ferritin (less than about 2930 ng/mL), and IgE (greater than about 300 ng/mL).

It is understood that these values are approximate. Statistical methods can be used to define the critical range of values. Typically within one standard deviation of those approximate values might be considered as statistically significant values for determining a statistically significant difference, preferably two standard deviations. For this reason, the word “about” is used in connection with the stated values. “Statistical classification methods” are used to identify biomarkers capable of discriminating normal patients from patients with galactosemia and are further used to determine critical blood values for each biomarker for discriminating between such patients. Certain statistical methods can be used to identify discriminating biomarkers and panels thereof. These statistical methods may include, but are not limited to: 1) linear regression; 2) classification tree methods; and 3) statistical machine learning to optimize the unbiased performance of algorithms for making predictions. Each of these statistical methods is well-known to those of ordinary skill in the field of biostatistics and can be performed as a process in a computer. A large number of software products are available commercially to implement statistical methods, such as, without limitation, S-PLUS™, commercially available from Insightful Corporation of Seattle, Wash.

By identifying biomarkers useful in the determination and/or diagnosis of galactosemia and by use of statistical methods to identify which biomarkers and groups of biomarkers are particularly useful in identifying galactosemia-at-risk patients, a person of ordinary skill in the art, based on the disclosure herein, can compose panels of biomarkers having superior selectivity and sensitivity. Examples of biomarkers that can be included in panels, which provide excellent discriminatory capability, include: Eotaxin, MCP-1, Alpha-2 Macroglobulin, Apolipoprotein H, Cancer Antigen 125, Leptin, TNF RII, Alpha-Fetoprotein, IgM, MIP-1-alpha, Ferritin, and IgE. Examples of specific panels comprising selected biomarkers from the above-mentioned list, include, but are not limited to: (i) Eotaxin, MIP-1 alpha, Leptin, and IgE (ii) MCP-1, Ferritin, TNF RII, and IgM and (iii) Eotaxin, MCP-1, MIP-1-alpha, and Ferritin. It will be recognized by those of ordinary skill in the field of biostatistics, that the number of biomarkers in any given panel may be different depending on the combination of biomarkers. With optimum sensitivity and specificity being the goal, one panel may include two biomarkers, another may include five, and still others may include twelve or more, yielding similar results.

The invention is based on an evaluation of at least Eotaxin levels, alone or in combination with levels of MCP-1 and/or other biomarkers, in serum for diagnosis of galactosemia in all stages of it progression. The invention is also based on the evaluation of at least MCP-1 levels, optionally in combination with levels of at least Eotaxin. Patients with galactosemia are at considerable risk for cataracts, cirrhosis, severe infection with bacteria (E. coli sepsis), delayed speech development, severe mental retardation, irregular menstrual cycles, decreased function of ovaries, leading to ovarian failure, tremors and uncontrollable motor functions, and death, if a galactose-free diet is not adhered to and serious complications, and outcomes can be improved with appropriate diagnosis and therapy.

The results described herein demonstrate that serum Eotaxin levels are depressed in galactosemia patients. Thus, Eotaxin can be used as an early biomarker of galactosemia. By the same token, MCP-1 levels are depressed in galactosemia. Thus, MCP-1 can be used as an early biomarker of galactosemia.

The present method includes measuring the level of Eotaxin and/or MCP-1 in a biological sample (e.g., whole blood, plasma, serum or urine and the like) from a patient; comparing the respective levels with that of control subjects; and diagnosing the state of disease based on the level of Eotaxin or MCP-1 relative to that of control subjects. A patient can be diagnosed with galacotsemia if the level of Eotaxin is decreased relative to that of control subjects or if MCP-1 is decreased relative to controls.

A typical control value for Eotaxin is in the range of about 370-500 pg/mL. A concentration of about 370 pg/mL or less in a patient sample supports a positive diagnosis. The general range for depressed values of Eotaxin is about 100-350 pg/mL.

A typical control value for MCP-1 is in the range of about 2-5 ng/mL. An immunological concentration of about 1 ng/mL in a patient sample supports a positive diagnosis.

Eotaxin and MCP-1 can be captured with anti-Eotaxin and anti-MCP-1 polyclonal antibodies, respectively, or with corresponding monoclonal antibodies. The diagnostic method may also include measuring the levels of one or more additional analytes selected from the group consisting of: Eotaxin, MCP-1, Alpha-2 Macroglobulin, Apolipoprotein H, Cancer Antigen 125, Leptin, TNF RII, Alpha-Fetoprotein, IgM, MIP-1-alpha, Ferritin, and IgE.

TABLE 1
Significant Analytes in Galactosemia Study

| | | Galactosemia | Control | P value |
|---|---|---|---|---|
| | Units | (Mean) | (Mean) | (t-test) |
| Eotaxin | pg/mL | 225 | 469 | 7.6E−06 |
| MCP-1 | ng/mL | 1.3 | 3.2 | 2.8E−04 |
| Alpha-2 Macrogobulin | mg/mL | 2.7 | 1.8 | 1.8E−03 |
| Apolipoprotein H | ug/mL | 47 | 67 | 6.3E−03 |
| Cancer Antigen 125 | U/mL | 7.8 | 4.2 | 1.1E−02 |
| Leptin | ng/mL | 1.2 | 0.46 | 1.2E−02 |
| TNF RII | ng/mL | 5.8 | 8.1 | 2.0E−02 |
| Alpha-Fetoprotein | ng/mL | 493 | 206 | 3.1E−02 |
| IgM | mg/mL | 0.21 | 0.055 | 3.5E−02 |
| MIP-1-alpha | ug/mL | 30 | 44 | 3.6E−02 |
| Ferritin | ng/mL | 2590 | 5201 | 4.3−02 |
| IgE | ng/mL | 345 | 225 | 4.5−02 |

Analyte levels can be measured using an immunoassay such as an ELISA or a multiplexed method as described below, and in more detail by Chandler et al., U.S. Pat. No. 5,981,180 (Luminex Corporation).

Eotaxin levels below 365 pg/mL were identified in galatosemic patients. Without being bound by a particular mechanism, Eotaxin may be directly involved in the pathophysiology of galactosemia. MCP-1 levels below 2 ng/mL were identified in galactosemic patients. The role MCP-1 plays in the pathophysiology of galactosemia, however, is not currently known.

The analytes used in the method of the invention can be detected, for example, by a binding assay. The term “binding reagent” and like terms, refers to any compound, composition or molecule capable of specifically or substantially specifically (that is with limited cross-reactivity) binding another compound or molecule, which, in the case of immune-recognition is an epitope. The binding reagents typically are antibodies, preferably monoclonal antibodies, or derivatives or analogs thereof, but also include, without limitation: Fᵥ fragments; single chain Fᵥ (scFᵥ) fragments; Fab′ fragments; F(ab′)₂ fragments; humanized antibodies and antibody fragments; camelized antibodies and antibody fragments; and multivalent versions of the foregoing. Multivalent binding reagents also may be used, as appropriate, including without limitation: mono-specific or bi-specific antibodies, such as disulfide stabilized Fᵥ fragments, scFᵥ tandems ((scFᵥ)₂ fragments), dibodies, tribodies or tetrabodies, which typically are covalently linked or otherwise stabilized (i.e., leucine zipper or helix stabilized) scFᵥ fragments. “Binding reagents” also include aptamers, as are described in the art.

Methods of making antigen-specific binding reagents, including antibodies and their derivatives and analogs and aptamers, are well-known in the art. Polyclonal antibodies can be generated by immunization of an animal. Monoclonal antibodies can be prepared according to standard (hybridoma) methodology. Antibody derivatives and analogs, including humanized antibodies can be prepared recombinantly by isolating a DNA fragment from DNA encoding a monoclonal antibody and subcloning the appropriate V regions into an appropriate expression vector according to standard methods. Phage display and aptamer technology is described in the literature and permit in vitro clonal amplification of antigen-specific binding reagents with very affinity low cross-reactivity. Phage display reagents and systems are available commercially, and include the Recombinant Phage Antibody System (RPAS), commercially available from Amersham Pharmacia Biotech, Inc. of Piscataway, N.J. and the pSKAN Phagemid Display System, commercially available from MoBiTec, LLC of Marco Island, Fla. Aptamer technology is described for example and without limitation in U.S. Pat. Nos. 5,270,163, 5,475,096, 5,840,867 and 6,544,776.

The ELISA and Luminex LabMAP immunoassays described below are examples of sandwich assays. The term “sandwich assay” refers to an immunoassay where the antigen is sandwiched between two binding reagents, which are typically antibodies. The first binding reagent/antibody being attached to a surface and the second binding reagent/antibody comprising a detectable group. Examples of detectable groups include, for example and without limitation: fluorochromes, enzymes, epitopes for binding a second binding reagent (for example, when the second binding reagent/antibody is a mouse antibody, which is detected by a fluorescently-labeled anti-mouse antibody), for example an antigen or a member of a binding pair, such as biotin. The surface may be a planar surface, such as in the case of a typical grid-type array (for example, but without limitation, 96-well plates and planar microarrays), as described herein, or a non-planar surface, as with coated bead array technologies, where each “species” of bead is labeled with, for example, a fluorochrome (such as the Luminex technology described herein and in U.S. Pat. Nos. 6,599,331, 6,592,822 and 6,268,222), or quantum dot technology (for example, as described in U.S. Pat. No. 6,306,610).

In the bead-type immunoassays described in the examples below, the Luminex LabMAP system is utilized. The LabMAP system incorporates polystyrene microspheres that are dyed internally with two spectrally distinct fluorochromes. Using precise ratios of these fluorochromes, an array is created consisting of 100 different microsphere sets with specific spectral addresses. Each microsphere set can possess a different reactant on its surface. Because microsphere sets can be distinguished by their spectral addresses, they can be combined, allowing up to 100 different analytes to be measured simultaneously in a single reaction vessel. A third fluorochrome coupled to a reporter molecule quantifies the biomolecular interaction that has occurred at the microsphere surface. Microspheres are interrogated individually in a rapidly flowing fluid stream as they pass by two separate lasers in the Luminex analyzer. High-speed digital signal processing classifies the microsphere based on its spectral address and quantifies the reaction on the surface in a few seconds per sample.

For the assays described herein, the bead-type immunoassays are preferable for a number of reasons. As compared to ELISAs, costs and throughput are far superior. As compared to typical planar antibody microarray technology (for example, in the nature of the BD Clontech Antibody arrays, commercially available form BD Biosciences Clontech of Palo Alto, Calif.), the beads are far superior for quantification purposes because the bead technology does not require pre-processing or titering of the plasma or serum sample, with its inherent difficulties in reproducibility, cost and technician time. For this reason, although other immunoassays, such as, without limitation, ELISA, RIA and antibody microarray technologies, are capable of use in the context of the present invention, but they are not preferred. As used herein, “immunoassays” refer to immune assays, typically, but not exclusively sandwich assays, capable of detecting and quantifying a desired blood biomarker, namely at least one of Eotaxin, MCP-1, Alpha-2 Macroglobulin, Apolipoprotein H, Cancer Antigen 125, Leptin, TNF RII, Alpha-Fetoprotein, IgM, MIP-1-alpha, Ferritin, and IgE or any combination of the foregoing.

Data generated from an assay to determine blood levels of one, two, three, or four or more of the biomarkers Eotaxin, MCP-1, Alpha-2 Macroglobulin, Apolipoprotein H, Cancer Antigen 125, Leptin, TNF RII, Alpha-Fetoprotein, IgM, MIP-1-alpha, Ferritin, and IgE can be used to determine the likelihood of a patient suffering from galactosemia. As shown herein, if any one or more, two or more, typically three or four or more of the following conditions are met in a patient's blood Eotaxin (less than about 370 pg/mL), MCP-1 (less than about 2 ng/mL), Alpha-2 Macroglobulin (greater than about 2.0 mg/mL), Apolipoprotein H (less than about 55 ug/mL), Cancer Antigen 125 (greater than about 5.0 U/mL), Leptin (greater than about 0.57 ng/mL), TNF RII (less than about 5.95 ng/mL), Alpha-Fetoprotein (less than about 330 ng/mL), IgM (greater than about 0.051 mg/mL), MIP-1 alpha (less than about 38 pg/mL), Ferritin (less than about 2930 ng/mL), and IgE (greater than about 300 ng/mL), there is a very high likelihood that the patient has suffered or is suffering from a galactosemia. In one embodiment, either an depressed Eotaxin level or a depressed MCP-1 level alone, relative to the level of the biomarker of interest in a population of normal or control patients, indicates the existence of galactosemia in the patient with about a 97-99% level of certainty. (See, Table 2, discussed further elsewhere herein.)

In the context of the present disclosure, “blood” includes any blood fraction, for example serum, that can be analyzed according to the methods described herein. Serum is a standard blood fraction that can be tested, and is tested in the Examples below. By measuring blood levels of a particular biomarker, it is meant that any appropriate blood fraction can be tested to determine blood levels and that data can be reported as a value present in that fraction. As a non-limiting example, the blood levels of a biomarker can be presented as 50 pg/mL serum.

### Example I

Development of Luminex assay. The reagents for multiplex system were developed using antibody pairs purchased from R&D Systems (Minneapolis, Minn.), Fitzgerald Industries International (Concord, Mass.) or produced by well known immunological methods. Capture antibodies were monoclonal and detection antibodies were polyclonal. Capture Abs were covalently coupled to carboxylated polystyrene microspheres number 74 purchased from Luminex Corporation (Austin, Tex.). Covalent coupling of the capture antibodies to the microspheres was performed by following the procedures recommended by Luminex. In short, the microspheres' stock solutions were dispersed in a sonification bath (Sonicor Instrument Corporation, Copiaque, N.Y.) for 2 min. An aliquot of 2.5×10⁶ microspheres was resuspended in microtiter tubes containing 0.1 M sodium phosphate buffer, pH 6.1 (phosphate buffer), to a final volume of 80 μL. This suspension was sonicated until a homogeneous distribution of the microspheres was observed. Solutions of N-hydroxy-sulfosuccinimide (Sulfo-NHS) and 1-ethyl-3-(3-dimethylaminopropyl)-carbodiimide hydrochloride (Pierce), both at 50 mg/mL, were prepared in phosphate buffer, and 10 μL of each solution was sequentially added to stabilize the reaction and activate the microspheres. This suspension was incubated for 10 min at room temperature and then resuspended in 250 μL of PBS containing 50 μg of antibody. The mixture was incubated overnight in the dark with continuous shaking. Microspheres were then incubated with 250 μL of PBS-0.05% Tween 20 for 4 h. After aspiration, the beads were blocked with 1 mL of PBS-1% BSA-0.1% sodium azide. The microspheres were counted with a hemacytometer and stored at a final concentration of 10⁶ microspheres per mL in the dark at 4 C. Coupling efficiency of monoclonal antibodies was tested by staining 2,000 microspheres with PE-conjugated goat anti-mouse IgG (BD Biosciences, San Diego, Calif.). Detection Abs were biotinylated using EZ-Link Sulfo-NHS-Biotinylation Kit (Pierce, Rockford, Ill.) according to manufacturer's protocol. The extent of biotin incorporation was determined using HABA assay and was 20 moles of biotin per mole of protein. The assays were further optimized for concentration of detection Ab and for incubation times. Sensitivity of the newly developed assays were determined using serially diluted purified proteins. Intra-assay variability, expressed as a coefficient of variation, was calculated based on the average for patient samples and measured twice at two different time points. The intra-assay variability within the replicates is expressed as an average coefficient of variation. Inter-assay variability was evaluated by testing quadruplicates of each standard and sample with an average of 16.5% (data not shown). Newly developed kits were multiplexed together and the absence of cross-reactivity was confirmed according to Luminex protocol.

Examples of some commercial sources of matched antibody cytokine pairs include MAB636 EGF (R&D Systems, Minneapolis, Minn.), BAF236 G-CSF (R&D Systems), DY214 IL-6 (R&D Systems), DY206 IL-8 (R&D Systems), DY208 IL-12p40 (R&D Systems), DY1240 MCP-1 (R&D Systems), DY279 VEGF (R&D Systems), DY293 CA-125 (M002201, M002203, Fitzgerald Industries International, Inc., Concord, Mass.).

### Results

Serum concentrations of biomarkers by LabMap technology. Circulating concentrations of different serum biomarkers were evaluated in a multiplexed assay using LabMap technology in blood of patients from galactosemic and control groups. Table 1 lists the analytes that are statistically different between the two groups.

Table 2 illustrates the diagnostic accuracy obtained by testing for each individual analyte and determining how useful it would be as a diagnostic tool.

TABLE 2
Analyte Accuracy for Galactosemia

| | | |
|---|---|---|
| Neg | Pos | Accuracy |

Eotaxin

| | | | |
|---|---|---|---|
| True Neg | 10 | 0 | |
| True Pos | | 10 | 100% |

MCP-1

| | | | |
|---|---|---|---|
| True Neg | 9 | 1 | |
| True Pos | 1 | 9 | 90% |

Alpha-2 Macroglobulin

| | | | |
|---|---|---|---|
| True Neg | 8 | 2 | |
| True Pos | 0 | 10 | 90% |

Apolipoprotein-H

| | | | |
|---|---|---|---|
| True Neg | 10 | 0 | |
| True Pos | 3 | 7 | 85% |

CA-125

| | | | |
|---|---|---|---|
| True Neg | 9 | 1 | |
| True Pos | 2 | 8 | 85% |

Leptin

| | | | |
|---|---|---|---|
| True Neg | 7 | 3 | |
| True Pos | 1 | 9 | 80% |

TNF-RII

| | | | |
|---|---|---|---|
| True Neg | 9 | 1 | |
| True Pos | 4 | 6 | 75% |

Alpha-Fetoprotein

| | | | |
|---|---|---|---|
| True Neg | 9 | 1 | |
| True Pos | 3 | 7 | 80% |

IgM

| | | | |
|---|---|---|---|
| True Neg | 10 | 0 | |
| True Pos | 4 | 6 | 80% |

MIP-1alpha

| | | | |
|---|---|---|---|
| True Neg | 7 | 3 | |
| True Pos | 4 | 6 | 65% |

Ferritin

| | | | |
|---|---|---|---|
| True Neg | 9 | 1 | |
| True Pos | 3 | 7 | 80% |

IgE

| | | | |
|---|---|---|---|
| True Neg | 7 | 3 | |
| True Pos | 2 | 8 | 75% |

Proximity Map Analysis. The proximity map data analysis is conducted with a software program that groups samples by their similarities in analyte concentration patterns. A unique chemical signature is generated using the concentration of the analytes measured in each sample. The relationship of each sample signature is visualized in the Galaxy™ projection. The Galaxy™ is a proximity map, such that the closer two objects are in the visualization, the closer their chemical signatures are, and thus the more similar they are to one another. The axes are dimensionless (a result of being derived from a principal component analysis), and thus the visualization is not a typical X-Y scatter plot in which moving along one axis means increasing or decreasing a single value. The two axes of the Galaxy™ are defined by the first two principal components, a common method to reduce complex data. The placement of objects (record points) is done using a set of heuristics that have been designed to maximize the preservation of spatial relationships that existed in the high-dimensional space of the original data while minimizing the overlap that can occur when doing simple projections.

Whereas particular embodiments of the invention have been described herein for the purpose of illustrating the invention and not for the purpose of limiting the same, it will be appreciated by those of ordinary skill in the art that numerous variations of the details, materials and arrangement of parts may be made within the principle and scope of the invention without departing from the invention as described in the appended claims.

What is claimed is:

1. A method of diagnosing galactosemia in a human subject suspected of suffering from galactosemia, comprising:
(a) obtaining a fluid sample from a human subject suspected of suffering from galactosemia;
(b) determining the concentration of Eotaxin in said fluid sample;
(c) deciding if the determined concentration of Eotaxin in said fluid sample is statistically different from that found in a control group of human subjects,
whereby a statistically different depressed concentration of Eotaxin supports a positive diagnosis of galactosemia.

2. The method of claim 1 in which said human subject is a newborn.

3. The method of claim 1 in which said fluid sample is selected from the group consisting of blood spots, whole blood, plasma, serum, or urine.

4. The method of claim 1 in which a determined concentration of about 365 pg/mL or below of Eotaxin in said fluid sample supports a positive diagnosis.

5. A method of diagnosing galactosemia in a human subject suspected of suffering from galactosemia, comprising:
(a) obtaining a fluid sample from a human subject suspected of suffering from galactosemia;
(b) determining the concentration of MCP-1 in said fluid sample;
(c) deciding if the determined concentration of MCP-1 in said fluid sample is statistically different from that found in a control group of human subjects,
whereby a statistically different depressed concentration of MCP-1 supports a positive diagnosis of galactosemia.

6. The method of claim 5 in which said human subject is a newborn.

7. The method of claim 5 in which said fluid sample is selected from the group consisting of blood spots, whole blood, plasma, serum, or urine.

8. The method of claim 5 in which a determined concentration of about 1 ng/mL or below of MCP-1 in said fluid sample supports a positive diagnosis.

9. A method of diagnosing galactosemia in a human subject suspected of suffering from galactosemia, comprising:
(a) obtaining a fluid sample from a human subject suspected of suffering from galactosemia;
(b) determining the concentrations of Eotaxin and MCP-1 in said fluid sample;
(c) deciding if the determined concentrations of Eotaxin and MCP-1 in said fluid sample are statistically different from that found in a control group of human subjects,
whereby a statistically different depressed concentration of Eotaxin and a statistically different depressed concentration of MCP-1 support a positive diagnosis of galactosemia.

10. The method of claim 9 which further comprises determining the concentration in said fluid sample of at least one of Alpha-2 Macroglobulin, Apolipoprotein H, Cancer Antigen 125, Leptin, TNF RII, Alpha-Fetoprotein, IgM, MIP-1-alpha, Ferritin, and IgE or any combination thereof.

11. The method of claim 10 in which statistically different concentrations, compared to control levels, of all analytes support a positive diagnosis of galactosemia.

12. The method of claim 9 in which concentrations are determined by conducting one or more immunoassays.

13. The method of claim 9 which further comprises determining the concentration in said fluid sample of at least one of Alpha-2 Macroglobulin, Apolipoprotein H, Cancer Antigen 125, Leptin, TNF RII, Alpha-Fetoprotein, IgM, MIP-1-alpha, Ferritin, and IgE or any combination thereof.

14. The method of claim 13 in which statistically different concentrations, compared to control levels, of Alpha-2 Macroglobulin, Apolipoprotein H, Cancer Antigen 125, Leptin, TNF RII, Alpha-Fetoprotein, IgM, MIP-1-alpha, Ferritin, and IgE support a positive diagnosis of galactosemia.

15. The method of claim 9 in which a subject's determined concentrations of analytes in said fluid sample are presented in a proximity map, whereby the proximity of a subject's determined concentrations to a cluster of other subjects' determined concentrations, who were previously diagnosed as having suffered from galactosemia, contributes to a positive diagnosis of galactosemia.

16. A kit comprising reagents for determining the concentration in a fluid sample of a panel of analytes including Eotaxin and MCP-1 and one or more of Alpha-2 Macroglobulin, Apolipoprotein H, Cancer Antigen 125, Leptin, TNF RII, Alpha-Fetoprotein, IgM, MIP-1-alpha, Ferritin, and IgE.

17. The kit of claim 16 which includes antibodies against a panel of analytes including Alpha-2 Macroglobulin, Apolipoprotein H, Cancer Antigen 125, Leptin, TNF RII, Alpha-Fetoprotein, IgM, MIP-1-alpha, Ferritin, and IgE.

18. The kit of claim 16 which includes reagents immobilized on a substrate.

19. The kit of claim 18 which the substrate comprises a two-dimensional array, a microtiter plate, or multiple bead sets.
